SEQUENCE LISTING (10 sequences)

SEQ ID NO: 1 - DNA, 4500 bp, feline infectious peritonitis coronavirus
  CDS (70)..(4428)
gaagggtaagttactcattagaaataatggcaagctactaaactttggtaatcatttagttaatgtgccatgattgtgctcgtaacttgcctcttgttgttatgttcataccacacagttttgagtacaacaaataatgaatgcatacaagttaacgtaacacaattggctggcaatgaaaaccttatcagagattttctgtttagtaactttaaagaagaaggaagtgtagttgttggtggttattaccctacagaggtgtggtacaactgctctagaacagctcgaactactgcctttcagtattttaataatatacatgccttttattttgttatggaagccatggaaaatagcactggtaatgcacgtggtaaaccattattatttcatgtgcatggtgagcctgttagtgttattatatcggcttatagggatgatgtgcaacaaaggccccttttaaaacatgggttagtgtgcataactaaaaatcgccatattaactatgaacaattcacctccaaccagtggaattccacatgtacgggtgctgacagaaaaattcctttctctgtcatacccacggacaatggaacaaaaatctatggtcttgagtggaatgatgactttgttacagcttatattagtggtcgttcttatcacttgaacatcaatactaattggtttaacaatgtcacacttttgtattcacgctcaagcactgctacctgggaatacagtgctgcatatgcttaccaaggtgtttctaacttcacttattacaagttaaataacaccaatggtctaaaaacctatgaattatgtgaagattatgaacattgcactggctatgctaccaatgtatttgctccgacatcaggtggttacatacctgatggatttagttttaacaattggttcttgcttacaaatagttccacttttgttagtggcaggtttgtaacaaatcaaccattattgattaattgcttgtggccagtgcccagttttggtgtagcagcacaagaattttgttttgaaggtgcacagtttagccaatgtaatggtgtgtctttaaataacacagtggatgttattagattcaaccttaatttcactgcagatgtacaatctggtatgggtgctacagtattttcactgaatacaacaggtggtgtcattcttgaaatttcatgttatagtgacacagtgagtgagtctagttcttacagttatggtgaaatcccgttcggcataactgacggaccacgatactgttatgtactttacaatggcacagctcttaaatatttaggaacattaccacccagtgtaaaggaaattgctattagtaagtggggccatttttatattaatggttacaatttctttagcacatttcctattggttgtatatcttttaatttaaccactggtgttagtggagctttttggacaattgcttacacatcgtatactgaagcattagtacaagttgaaaacacagctattaaaaatgtgacgtattgtaacagtcacattaataacattaaatgttctcaacttactgctaatttgaataatggattttatcctgttgcttcaagtgaagtaggtttcgttaataagagtgttgtgttattacctagctttttcacatacaccgctgtcaatataaccattgatcttggtatgaagcttagtggttatggtcaacccatagcctcgacactaagtaacatcacactaccaatgcaggataacaatactgatgtgtactgtattcgttctaaccaattctcagtttatgttcattccacttgcaaaagttctttatgggacaatatttttaatcaagactgcacggatgttttagaggctacagctgttataaaaactggtacttgtcctttctcatttgataaattgaacaattacttgacttttaacaagttctgtttgtcgttgagtcctgttggtgctaattgcaagtttgatgttgctgcacgtacaagaaccaatgagcaggttgttagaagtctatatgtaatatatgaagaaggagacaacatagtgggtgtaccgtctgataatagcggtctgcacgatttgtctgtgctacacctagactcctgtacagattacaatatatatggtagaactggtgttggtattattagacgaactaacagtacgctacttagtggcttatattacacatcactatcaggtgatttgttaggctttaaaaatgttagtgatggtgtcatttattctgtgacgccatgtgatgtaagcgcacaagcggctgttattgatggtgccatagttggagctatgacttccattaacagtgaactgttaggtctaacacattggacaacgacacctaatttttattactactctatatataattacacaagtgagaggactcgtggcactgcaattgacagtaacgatgttgattgtgaacctgtcataacctattctaatataggtgtttgtaaaaatggtgctttggtttttattaacgtcacacattctgacggagacgtgcaaccaattagcactggtaatgtcacgatacctacaaattttactatatctgtgcaagttgaatacatgcaggtttacactacaccagtatcaatagattgtgcaagatacgtttgtaatggtaaccctagatgtaacaaattgttaacacaatatgtgtctgcatgtcaaactattgaacaagcacttgcaatgggtgccagacttgaaaacatggaggttgattccatgttgtttgtctcggaaaatgcccttaaattggcatctgttgaggcgttcaatagtacagaaaatttagatcctatttacaaagaatggcctagcataggtggttcttggctaggaggtctaaaagatatactaccgtcccataatagcaaacgtaagtatggttctgctatagaagatttgctttttgataaagttgtaacatctggtttaggtacagttgatgaagattataaacgttgtactggtggttacgacatagcagacttggtgtgtgctcaatattacaatggcatcatggttctaccaggtgtagctaatgctgacaagatgactatgtacacagcatcacttgcaggtggtataacattaggtgcacttggtggtggcgccgtggctataccttttgcagtagcagtacaggctagacttaattatgttgctctacaaactgatgtattgaataaaaaccaacagatcctggctaatgctttcaatcaagctattggtaacattacacaggcttttggtaaggttaatgatgctatacatcaaacatcacaaggtcttgccactgttgctaaagcgttggcaaaagtgcaagatgttgtcaacacacaagggcaagctttaagtcaccttacagtacaattgcaaaataattttcaagccattagtagttctattagtgatatttataacaggcttgacgaactgagtgctgatgcacaagttgataggctgattacaggtagacttacagcacttaatgcatttgtgtctcagactctaaccagacaagcagaggttagggctagtagacaacttgccaaagacaaggttaatgaatgtgttaggtctcagtctcagagattcggattctgtggtaatggtacacatttgttttcactagcaaatgcagcaccaaatggcatgattttctttcatacagtactattaccaacagcttatgaaactgtaacagcttggtcaggtatttgtgcttcagatggcgatcgcactttcggacttgtcgttaaagatgtgcagttgacgttgtttcgtaatctagatgacaagttctatttgacccccagaactatgtatcagcctagagttgcaactagttctgattttgttcaaattgaagggtgtgatgtgttgtttgtcaacgcgactgtaattgatttgcctagtattatacctgactatattgacattaatcaaactgttcaagacatattagaaaattacagaccaaactggactgtacctgaatttacacttgatattttcaacgcaacctatttaaatctgactggtgaaattgatgacttagagtttaggtcagaaaagctacataacactacagtagaacttgccattctcattgataacattaataatacattagtcaatcttgaatggctcaatagaattgaaacttatgtaaaatggccttggtatgtgtggctactgataggtttagtagtagtattttgcataccattactgctattttgctgttttagcacaggttgttgtggatgcataggttgtttaggaagttgttgtcactctatatgtagtagaagacaatttgaaaattatgaaccaattgaaaaagtgcatgtccactaaatttaaagttaaggatgttgaataaattccttaagaactaaacttattagtcattacaggtcttgtatggac

SEQ ID NO: 2 - protein, 1452 aa, feline infectious peritonitis coronavirus
MIVLVTCLLLLCSYHTVLSTTNNECIQVNVTQLAGNENLIRDFLFSNFKEEGSVVVGGYYPTEVWYNCSRTARTTAFQYFNNIHAFYFVMEAMENSTGNARGKPLLFHVHGEPVSVIISAYRDDVQQRPLLKHGLVCITKNRHINYEQFTSNQWNSTCTGADRKIPFSVIPTDNGTKIYGLEWNDDFVTAYISGRSYHLNINTNWFNNVTLLYSRSSTATWEYSAAYAYQGVSNFTYYKLNNTNGLKTYELCEDYEHCTGYATNVFAPTSGGYIPDGFSFNNWFLLTNSSTFVSGRFVTNQPLLINCLWPVPSFGVAAQEFCFEGAQFSQCNGVSLNNTVDVIRFNLNFTADVQSGMGATVFSLNTTGGVILEISCYSDTVSESSSYSYGEIPFGITDGPRYCYVLYNGTALKYLGTLPPSVKEIAISKWGHFYINGYNFFSTFPIGCISFNLTTGVSGAFWTIAYTSYTEALVQVENTAIKNVTYCNSHINNIKCSQLTANLNNGFYPVASSEVGFVNKSVVLLPSFFTYTAVNITIDLGMKLSGYGQPIASTLSNITLPMQDNNTDVYCIRSNQFSVYVHSTCKSSLWDNIFNQDCTDVLEATAVIKTGTCPFSFDKLNNYLTFNKFCLSLSPVGANCKFDVAARTRTNEQVVRSLYVIYEEGDNIVGVPSDNSGLHDLSVLHLDSCTDYNIYGRTGVGIIRRTNSTLLSGLYYTSLSGDLLGFKNVSDGVIYSVTPCDVSAQAAVIDGAIVGAMTSINSELLGLTHWTTTPNFYYYSIYNYTSERTRGTAIDSNDVDCEPVITYSNIGVCKNGALVFINVTHSDGDVQPISTGNVTIPTNFTISVQVEYMQVYTTPVSIDCARYVCNGNPRCNKLLTQYVSACQTIEQALAMGARLENMEVDSMLFVSENALKLASVEAFNSTENLDPIYKEWPSIGGSWLGGLKDILPSHNSKRKYGSAIEDLLFDKVVTSGLGTVDEDYKRCTGGYDIADLVCAQYYNGIMVLPGVANADKMTMYTASLAGGITLGALGGGAVAIPFAVAVQARLNYVALQTDVLNKNQQILANAFNQAIGNITQAFGKVNDAIHQTSQGLATVAKALAKVQDVVNTQGQALSHLTVQLQNNFQAISSSISDIYNRLDELSADAQVDRLITGRLTALNAFVSQTLTRQAEVRASRQLAKDKVNECVRSQSQRFGFCGNGTHLFSLANAAPNGMIFFHTVLLPTAYETVTAWSGICASDGDRTFGLVVKDVQLTLFRNLDDKFYLTPRTMYQPRVATSSDFVQIEGCDVLFVNATVIDLPSIIPDYIDINQTVQDILENYRPNWTVPEFTLDIFNATYLNLTGEIDDLEFRSEKLHNTTVELAILIDNINNTLVNLEWLNRIETYVKWPWYVWLLIGLVVVFCIPLLLFCCFSTGCCGCIGCLGSCCHSICSRRQFENYEPIEKVHVH

SEQ ID NO: 3 - protein, 21 aa, feline infectious peritonitis coronavirus
GGSWLGGLKDILPSHNSKRKY

SEQ ID NO: 4 - protein, 59 aa, feline infectious peritonitis coronavirus
HNSKRKYGSAIEDLLFDKVVTSGLGTVDEDYKRCTGGYDIADLVCAQYYNGIMVLPGVA

SEQ ID NO: 5 - protein, 31 aa, feline infectious peritonitis coronavirus
MYQPRVATSSDFVQIEGCDVLFVNATVIDLP

SEQ ID NO: 6 - protein, 365 aa, feline infectious peritonitis coronavirus
GGSWLGGLKDILPSHNSKRKYGSAIEDLLFDKVVTSGLGTVDEDYKRCTGGYDIADLVCAQYYNGIMVLPGVANADKMTMYTASLAGGITLGALGGGAVAIPFAVAVQARLNYVALQTDVLNKNQQILANAFNQAIGNITQAFGKVNDAIHQTSQGLATVAKALAKVQDVVNTQGQALSHLTVQLQNNFQAISSSISDIYNRLDELSADAQVDRLITGRLTALNAFVSQTLTRQAEVRASRQLAKDKVNECVRSQSQRFGFCGNGTHLFSLANAAPNGMIFFHTVLLPTAYETVTAWSGICASDGDRTFGLVVKDVQLTLFRNLDDKFYLTPRTMYQPRVATSSDFVQIEGCDVLFVNATVIDLP

SEQ ID NO: 7 - DNA, 63 bp, feline infectious peritonitis coronavirus
ggtggttcttggctaggaggtctaaaagatatactaccgtcccataatagcaaacgtaagtat

SEQ ID NO: 8 - DNA, 177 bp, feline infectious peritonitis coronavirus
cataatagcaaacgtaagtatggttctgctatagaagatttgctttttgataaagttgtaacatctggtttaggtacagttgatgaagattataaacgttgtactggtggttacgacatagcagacttggtgtgtgctcaatattacaatggcatcatggttctaccaggtgtagct

SEQ ID NO: 9 - DNA, 93 bp, feline infectious peritonitis coronavirus
atgtatcagcctagagttgcaactagttctgattttgttcaaattgaagggtgtgatgtgttgtttgtcaacgcgactgtaattgatttgcct

SEQ ID NO: 10 - DNA, 1095 bp, feline infectious peritonitis coronavirus
ggtggttcttggctaggaggtctaaaagatatactaccgtcccataatagcaaacgtaagtatggttctgctatagaagatttgctttttgataaagttgtaacatctggtttaggtacagttgatgaagattataaacgttgtactggtggttacgacatagcagacttggtgtgtgctcaatattacaatggcatcatggttctaccaggtgtagctaatgctgacaagatgactatgtacacagcatcacttgcaggtggtataacattaggtgcacttggtggtggcgccgtggctataccttttgcagtagcagtacaggctagacttaattatgttgctctacaaactgatgtattgaataaaaaccaacagatcctggctaatgctttcaatcaagctattggtaacattacacaggcttttggtaaggttaatgatgctatacatcaaacatcacaaggtcttgccactgttgctaaagcgttggcaaaagtgcaagatgttgtcaacacacaagggcaagctttaagtcaccttacagtacaattgcaaaataattttcaagccattagtagttctattagtgatatttataacaggcttgacgaactgagtgctgatgcacaagttgataggctgattacaggtagacttacagcacttaatgcatttgtgtctcagactctaaccagacaagcagaggttagggctagtagacaacttgccaaagacaaggttaatgaatgtgttaggtctcagtctcagagattcggattctgtggtaatggtacacatttgttttcactagcaaatgcagcaccaaatggcatgattttctttcatacagtactattaccaacagcttatgaaactgtaacagcttggtcaggtatttgtgcttcagatggcgatcgcactttcggacttgtcgttaaagatgtgcagttgacgttgtttcgtaatctagatgacaagttctatttgacccccagaactatgtatcagcctagagttgcaactagttctgattttgttcaaattgaagggtgtgatgtgttgtttgtcaacgcgactgtaattgatttgcct

(19) United States
(12) Patent Application Publication
(10) Pub. No.: US 2005/0053622 A1
(43) Pub. Date: Mar. 10, 2005

(54) Anti-coronavirus vaccine

(76) Inventors: Andre Aubert, Antibes (FR); Veronique Duquesne, Carros (FR); Marc Eloit, Saint-Maur (FR); Valerie Gonon, Le Perray en Yvelines (FR)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/485,258

(22) PCT Filed: Aug. 9, 2002
(86) PCT No.: PCT/FR02/02843

(30) Foreign Application Priority Data
Aug. 9, 2001 (FR) 0110644

(51) Int. Cl. A61K 39/12; C07K 14/005

(52) U.S. Cl. 424/204.1; 530/350

(57) ABSTRACT

The invention relates to a vaccine against coronavirus infections and, in particular, against feline infectious peritonitis (FIP). The inventive vaccine comprises immunogenic peptides included in the S protein of feline coronaviruses (FcoV), which do not result from immunologic enhancement. The invention also relates to the use of at least one peptide for the preparation of a vaccine that induces protection against coronavirus infections, said peptide being selected from the group comprising fragments of an S protein of coronavirus of at least 12 amino acids, included in the SEQ ID NO: 5, or nucleic acid fragments of at least 36 nucleotides, included in the SEQ ID NO: 10 and coding for one of said peptides.

The present invention relates to a vaccine against coronavirus infections, and in particular against feline infectious peritonitis (PIF), comprising immunogenic peptides contained in the feline coronavirus (FCoV) S protein, which do not induce an enhancement phenomenon.

Feline infectious peritonitis is a systemic disease, which is most often fatal, in wild and domestic cats. The agent responsible is a coronavirus, the feline infectious peritonitis virus (FIPV), which belongs to an antigenic group which comprises in particular feline enteric coronavirus (FECV), canine coronavirus (CCV), swine transmissible gastroenteritis coronavirus (TGEV), porcine respiratory coronavirus (PRCV) and human coronavirus (HCV), and which induces, in a host-dependent manner, a range of symptoms which range from mild enteritis to the severe debilitating disease, and, in some cases, up to death.

Feline coronavirus, like other coronaviruses, is a single-stranded RNA virus, of positive polarity, having approximately 30 kilobases. This virus is enveloped and comprises peplomeric structures called “Spikes”, spicules or S protein. The 3′ end of the RNA encodes in particular the following structural proteins:
a membrane protein, also called matrix (M) protein. The M protein is the most abundant membrane glycoprotein (25-30 kDa). Only 10% of the N-terminal part of the molecule is exposed at the surface of the virion. It appears to be important for the viral maturation of coronaviruses and for determining the site where the viral particles are assembled.
a nucleocapsid (N) protein. The N protein, which is also a glycoprotein (45-50 kDa), is the most highly conserved among the structural proteins of coronaviruses and it is necessary for encapsidating the genomic RNA and in particular for directing its incorporation into the capsid. This protein is probably involved in the replication of RNA.
another membrane protein, the sM (small membrane) protein is also known by the name of E (envelope) protein (about 10 kDa). It is a transmembrane protein which, like the M protein, plays a crucial role by forming a complete matrix for the spherical architecture of the virion and its budding, and
the abovementioned S protein, which is also a membrane protein which exists in the form of Spikes or spicules (S) and is also designated by the name of peplomer protein. The S protein is a glycoprotein (200-220 kDa), of which a large portion (spicules) is situated outside the viral envelope. It is responsible for the attachment of the virus to the host cell receptors (amino-peptidase N) and for the induction of the fusion of the viral envelope with the cell membrane.

FIPV, which is morphologically and antigenically very close to FECV, is distinguishable from the latter in that it has acquired the capacity to replicate in the macrophages [Pedersen, 1995]. In most cases, cats which have developed antibodies against FCoV, develop a disease which is more severe and more rapid, after the virulent challenge, than seronegative cats (absence of anti-FCoV antibodies). This enhancement phenomenon is due to the formation of viral infection enhancing antibodies directed against the S protein [Pedersen, 1980; Olsen et al., 1992], whose action has an effect which is deleterious and opposite to that of protecting antibodies, by forming immune complexes which are more infectious, particularly for macrophages; this phenomenon, which is also called ADE, for “antibody-dependent enhancement”, probably explains, at least in part, the low efficacy of vaccines comprising the coronavirus S protein.

In such a context, the development of a safe and effective vaccine against feline coronaviruses is therefore very problematic.

Now, feline infectious peritonitis (FIP) poses a major veterinary health problem; indeed, young cats are particularly sensitive to FIP: 54% of all FIP cases involve cats under 12 months old and 70% involve cats under 4 years old. Among these infected cats, infections caused by type I coronavirus strains appear to be predominant, whereas the type II infections represent respectively 5% and 20 to 30% of the FIP cases in the United States and in Japan [Pedersen et al., 1983; Hohdatsu et al., 1992].

Both serotypes are characterized on the basis of in vitro neutralization. The sera of cats affected by type I FCoVs neutralize other type I FCoVs, but not the type II FCoVs, and conversely. It is thought that the origin of the type II feline coronaviruses is RNA recombination events during which the gene for canine coronavirus S protein was incorporated into the genome of the type I FCoVs [Herrewegh et al., 1998].

According to the symptoms observed, two forms of the disease have been described:
an effusive or wet form, in which an accumulation of ascitic fluid is observed, caused by an intense inflammatory response and the activation of the complement cascade (increasing vascular permeability) [Pedersen, 1976; Pedersen, 1980], and
a non effusive or dry form, which is characterized by little or no accumulation of ascitic fluid, but in which granular fibrinous deposits are generally observed on various organs (liver, spleen, intestine, lungs). The results of biochemical and hematological analyses most often show: anemia, neutrophilia, lymphopenia, an increase in the total serum proteins and hyperglobulinemia accompanied by a decrease in the albumin level.

In the absence of effective treatments, various anti-FIPV vaccines have been proposed which use distinct vaccine strategies, taking into consideration the number and the type of antigens used and/or the mode and the form of expression and presentation of the antigens.

There may be mentioned for example vaccines incorporating inactivated whole viruses [Pedersen, 1983], attenuated live viruses or heterologous live viruses (CCV, HCV-229E, TGEV), but also vector-based vaccines based on poxviruses, the feline herpesvirus (FHV) or the adenovirus, which express the S, M or N protein or simple fragments of these proteins, in combination or fused with other carrier proteins [Vennema et al., 1991; EP 652 287; WO 97/20054; WO 97/20059; Woobs, R. D. et al., 1979; Stoddart, C. A. et al., 1988; Barlough, J. E. et al., 1985].

However, such vaccines, when they use the S protein, can induce enhancement phenomena by producing enhancing antibodies, which are incompatible with the establishment of an effective protection.

Other routes for the search for vaccines have also been proposed, in order to eliminate enhancing antibodies and thus to try to induce an effective humoral immunity.

They are, for the majority, recombinant subunit vaccines comprising, separately or in combination, at least one normal, recombinant or modified S, M or N protein or at least one peptide contained in one of these proteins [WO 97/20054; WO 92/08487]. More precisely:
the antigen may consist of an S protein modified at the sites or epitopes specifically associated with the ADE, at the N-terminal part of the S protein, that is to say more specifically: the A1 site (aa 562-598), the A2 site (aa 637-662) and possibly the D site [Corapi, W. V. et al., 1995; WO 95/07987; WO 96/06934; WO 95/07987; WO 93/23421]; however, this strategy has in particular the disadvantage of being based on mutations which are known to have the risk of causing a loss of antigenicity of the S protein as a whole;
the antigen may consist of a highly conserved region or UCD (Universal Conserved Domain), consisting of the C-terminal part of 124 amino acids (residues 1077 to 1276) of the S protein [WO 92/08487; WO 93/23421]. The determination of a universal sequence between various coronaviruses effectively shows the important role thereof in the structure of the protein and hence in the structure of the virus and/or in its replication. However, this does not at all make it possible to predict its immunoprotective activity; however, this fragment can induce the production of enhancing antibodies.

Also, these various strategies can induce a loss of antigenicity.

Although the exact reasons for which some of the apparently promising antigens give relatively weak efficiencies remain to be determined; they are likely to be multifactorial. Among the most critical factors is the type of antigen(s) or epitope(s). If an appropriate epitope is considered, cellular immunity is presumed to be the key which brings about protection (although there are a few studies which demonstrate that the neutralizing responses of antibodies could be the principal key to in vivo protection). Moreover, the antigen should be sufficiently immunogenic and capable of remaining in the presence of other antigens, whether competing or interfering antigens, such as for example other coronavirus proteins which would be necessary for the protection or antigens derived from other vaccines or combination of vaccines.

While the antigenic strength may be enhanced and while the antigenic competition can be overcome by various methods, compositions or combinations thereof, the protective nature of an antigen or an epitope, which are inherent to the amino acid sequence of the epitope, remain invariable, hence the need for a judicious choice of the antigen(s).

Consequently, none of the vaccine routes previously proposed has all the conditions required, namely:
the obtaining of a protective humoral immunity in vivo,
the absence of production of enhancing antibodies, and
the absence of enhancement phenomena in vivo.

The applicant consequently set itself the aim of producing a vaccine which is more suitable for the requirements of practical use, in particular in that it does not induce the formation of deleterious antibodies, while preserving the capacity to produce a protective immune response.

The applicant has selected peptides contained in the FIP coronavirus S protein, which, surprisingly, effectively make it possible to induce protective immunity, without inducing enhancement phenomena.

To obtain such a vaccine, the approach which consists in searching for the epitope sites which can be generally deduced by methods based on computer modeling or by extrapolating the antigenicity of the S protein, starting with other coronavirus species, or by a mixture of the two methods, allowing the localization of the “most probable” sequences, does not make it possible to deduce which peptides do not possess the deleterious properties.

The applicant has consequently developed a system which has made it possible to select particularly efficient peptides contained in the S protein.

The selection of these peptides is thus linked, surprisingly, to the resistance and/or to the sensitivity of cats to coronavirus infections, on the one hand, and to the regression or the progression of FIP, on the other hand.

In particular, the applicant has shown, surprisingly, that the selected peptides were specifically recognized by the sera from spontaneously regressing (SR) cats.

The subject of the present invention is thus the use of at least one peptide selected from the group consisting of the fragments of a coronavirus S protein of at least 12 amino acids, contained in SEQ ID NO: 5, excluding the fragments contained in the sequence corresponding to positions 175-298 of said SEQ ID NO: 5 or of the nucleic acid fragments of at least 36 nucleotides, contained in SEQ ID NO: 10, excluding the fragments contained in the sequence corresponding to positions 523-894 of said SEQ ID NO: 10 and encoding one of said peptides, for the preparation of a vaccine for inducing an exclusively neutralizing protection against coronavirus infections and in particular for protecting cats against feline infectious peritonitis (FIP).

In accordance with said use, said S protein fragments comprise between 12 and 20 amino acids.

The data below summarize the correspondence between the different sequences, for greater clarity:
SEQ ID NO: 6 according to the invention comprises 1452 amino acids and corresponds to the complete sequence of the S protein of the coronavirus FIPV 79-1146 strain;
SEQ ID NO: 5 according to the invention comprises 365 amino acids and corresponds to positions 940-1304 of the sequence SEQ ID NO: 6;
the peptide of SEQ ID NO: 2 according to the invention comprises 21 amino acids and corresponds to positions 1-21 of SEQ ID NO: 5 and to positions 940-960 of SEQ ID NO: 6;
the peptide of SEQ ID NO: 3 according to the invention comprises 59 amino acids and corresponds to positions 15-73 of SEQ ID NO: 5 and to positions 954-1012 of SEQ ID NO: 6;
the peptide of SEQ ID NO: 4 of 31 amino acids corresponds to positions 335-365 of SEQ ID NO: 5 and to positions 1274-1304 of SEQ ID NO: 6.

With reference to SEQ ID NO: 5 and NO: 6 according to the invention, the C-terminal fragment containing the universal domain (UCD) and the universal domain UCD of 124 amino acids, described in international application WO 93/23421, correspond to the following positions of SEQ ID NO: 5 and 6:

| Fragments of WO | Positions in | Positions in |
|---|---|---|
| 93/23421 | SEQ ID NO: 5 | SEQ ID NO: 6 |
| C-terminal fragment | Positions 138-337 | Positions |
| of the S protein | | 1077-1276 |
| (SEQ ID NO: 1-12 of | | |
| 199 or 200 amino | | |
| acids) | | |
| UCD fragment of 124 | Positions 175-298 | Positions |
| amino acids | | 1114-1237 |
| (positions 37-160 | | |
| of SEQ ID NO: 1-12) | | |

The subject of the present invention is also peptides consisting of a fragment of coronavirus S protein, characterized in that they do not induce enhancement phenomena, and in that they are obtained with the aid of the method comprising at least the following steps:
construction of a random peptide library corresponding to a coronavirus protein fragment, from at least one coronavirus viral genome,
bringing said peptides into contact with a spontaneously regressing (SR) cat serum, after infection with coronavirus, and
immunoselection of the peptides interacting with said spontaneously regressing cat sera, but scarcely or not even interacting at all with sera from cats exhibiting clinical symptoms (CS) or cats exhibiting subclinical signs of chronic infection (CI) with the coronavirus.

According to an advantageous embodiment of said peptides, they are selected from the group consisting of the peptide SEQ ID NO: 2, the peptides containing from 12 to 20 amino acids of the sequence SEQ ID NO: 2, the peptide SEQ ID NO: 3, the peptides containing from 12 to 20 amino acids of the sequence SEQ ID NO: 3, the peptide SEQ ID NO: 4 and the peptides containing from 12 to 20 amino acids of the sequence SEQ ID NO: 4.

Said peptides are contained between amino acids 940 and 1304 of the S protein, with reference to the S protein of SEQ ID NO: 6 of the FIPV 79-1146 strain and are therefore selected from the group consisting of peptide 7I corresponding to positions 940-960 (SEQ ID NO: 2) of the S protein of SEQ ID NO: 6, the peptides containing from 12 to 20 amino acids and whose sequence is contained in SEQ ID NO: 2, the peptide T12 corresponding to positions 954-1012 (SEQ ID NO: 3) of the S protein of SEQ ID NO: 6, the peptides containing from 12 to 20 amino acids and whose sequence is contained in SEQ ID NO: 3, the peptide 14I corresponding to positions 1274-1304 (SEQ ID NO: 4) of the S protein of SEQ ID NO: 6 and the peptides containing from 12 to 20 amino acids and whose sequence is contained in SEQ ID NO: 4. Surprisingly, said peptides:
do not induce enhancement phenomena, and
are capable of inducing immunity against coronavirus infections, and in particular against feline infectious peritonitis in cats.

None of the peptides according to the invention, that is to say exhibiting the properties of inducing neutralizing antibodies and of absence of induction of enhancing antibodies includes the fragment corresponding to positions 175-298 of SEQ ID NO: 5 or 1114-1237 of SEQ ID NO: 6, which can induce the production of enhancing antibodies.

In addition, the immunological properties of said peptides are correlated with the immunoprotecting reactions in a group of cats showing spontaneous regression of coronavirus infection.

Among said peptides, peptide T12 (SEQ ID NO: 3) is particularly preferred.

The invention also comprises the peptides, as defined above, modified by artificial mutations, deletions, insertions, variations or combinations of these events, provided that the peptides thus modified do not induce enhancement phenomena.

The invention also includes the peptides as defined above, in the form of synthetic peptides, of repeating peptides, of proteins fused at the N- or C-terminal end with a coronavirus protein (such as M, N, E) or with a protein of another feline (such as FIV, FeLV, FHV, Calicivirus, Parvovirus, Bordetella, Chlamidia), porcine (such as PCRV, parvovirus, chigger) or canine (such as CPV, Carre, CPI, rabies virus, A2/A1 virus, Babesia, leptospira, Lyme) pathogenic agent.

The subject of the present invention is also a vaccine for inducing protection against coronavirus infections and in particular for protecting cats against feline infectious peritonitis (FIP), characterized in that it comprises at least one peptide as defined above in combination with carrier substances and/or adjuvants and/or at least one pharmaceutically acceptable vehicle.

The adjuvants used are adjuvants which are conventionally used; advantageously, they are chosen from the group consisting of oily emulsions, saponin, inorganic substances, bacterial extracts, aluminum hydroxide and squalene.

The carrier substances are advantageously selected from the group consisting of unilamellar liposomes, multilamellar liposomes, saponin micelles or solid microspheres of a saccharide or auriferous nature.

According to an advantageous embodiment of said vaccine, it additionally comprises other appropriate viral proteins or peptides.

The subject of the present invention is also nucleic acid molecules encoding the various peptides as defined above.

More precisely, said nucleic acid molecules are selected in particular from the group consisting of the sequences SEQ ID NO: 7-10 and the nucleotide sequences containing from 36 to 60 nucleotides and whose sequence is contained in SEQ ID NO: 8 and the nucleotide sequences containing from 36 to 60 nucleotides and whose sequence is contained in SEQ ID NO: 9.

The subject of the present invention is also the use of said nucleic acid molecules for the construction of recombinant vectors (viruses or plasmids), which are useful as vaccines.

The subject of the present invention is also recombinant vectors, characterized in that they comprise a nucleic acid molecule, as defined above.

Advantageously, said vectors are preferably selected from the group consisting of viral vectors, such as poxviruses, adenoviruses, retroviruses, herpesviruses, bacterial vectors, such as mycobacteria, enterobacteria or lactobacilli and/or plasmids containing a sequence encoding at least one of the peptides as defined above. The subject of the present invention is also a vaccine for inducing protection against coronavirus infections and in particular for protecting cats against feline infectious peritonitis (FIP), characterized in that it comprises at least one nucleic acid molecule as defined above or a recombinant vector as defined above.

The immunogenic peptides of the S protein, as defined above, or the recombinant vectors expressing said peptides are particularly suitable for the prophylaxis of diseases caused by coronaviruses, including in particular FIPV.

Advantageously, said peptides may be obtained by chemical synthesis or by recombination of the corresponding DNA in a bacterium, a virus, a yeast or a eukaryotic host.

The vaccines according to the invention are capable of inducing a protective immune response against coronavirus diseases in dogs (CCV) and/or pigs (TGEV) and/or humans.

The vaccines according to the invention are advantageously administered systemically (intramuscularly, subcutaneously, intraperitoneally or intravenously) and/or locally (orally, nasally, or by other mucosal routes) or by a combination of these routes and effectively induce a protective immune response against coronaviruses, in particular against feline coronaviruses (FECV, FIPV).

The subject of the present invention is also a method for selecting immunogenic peptides corresponding to a fragment of a coronavirus protein, and not inducing enhancement phenomena, characterized in that it comprises at least the following steps:
construction of a random peptide library corresponding to a coronavirus protein fragment, from at least one coronavirus viral genome,
bringing said peptides into contact with at least one spontaneously regressing (SR) cat serum, after infection with coronavirus, and
immunoselection of the peptides interacting with said spontaneously regressing cat serum, but scarcely or not even interacting at all with sera from cats exhibiting clinical symptoms (CS) or cats exhibiting subclinical signs of chronic infection (CI) with a coronavirus.

Advantageously, the method consists in selecting immunogenic peptides corresponding to a fragment of a coronavirus S protein, and not inducing enhancement phenomena, characterized in that it comprises at least the following steps:
construction of a random peptide library corresponding to a coronavirus S protein fragment, from at least one FIPV viral genome,
bringing said peptides into contact with at least four different spontaneously regressing (SR) cat sera, after infection with FIPV, at a dilution of at least 1/1000, and
immunoselection of the peptides interacting with said spontaneously regressing cat sera, but scarcely or not even interacting at all with sera from cats exhibiting clinical symptoms (CS) or sera from cats exhibiting subclinical signs of chronic infection (CI) with FIPV.

Such a method allows the identification of the epitopes which are specifically correlated with protective antibody responses acquired in spontaneously regressing (SR) cats compared with the non protective immune reactions observed in the groups of cats exhibiting clinical symptoms (CS) or subclinical signs of chronic infection (CI) with FIPV [Gonon et al., 1999].

In such a method:
the use of four sera constitutes a statistically acceptable protocol;
the selected dilution makes it possible to achieve a specificity which could not be achieved with lower dilutions (cross-reactions).

The subject of the present invention is in addition a peptide consisting of a coronavirus S protein fragment, characterized in that it can be selected with the aid of the selection method as defined above and in that it is selected from the group consisting of the peptide SEQ ID NO: 2, the peptides containing from 12 to 20 amino acids and whose sequence is contained in SEQ ID NO: 2, the peptide SEQ ID NO: 3, the peptides containing from 12 to 20 amino acids and whose sequence is contained in SEQ ID NO: 3, the peptide SEQ ID NO: 4 and the peptides containing from 12 to 20 amino acids and whose sequence is contained in SEQ ID NO: 4.

In addition to the preceding features, the invention further comprises other features which will emerge from the description which follows, which refers to examples of carrying out the method which is the subject of the present invention and to the accompanying drawing, in which:

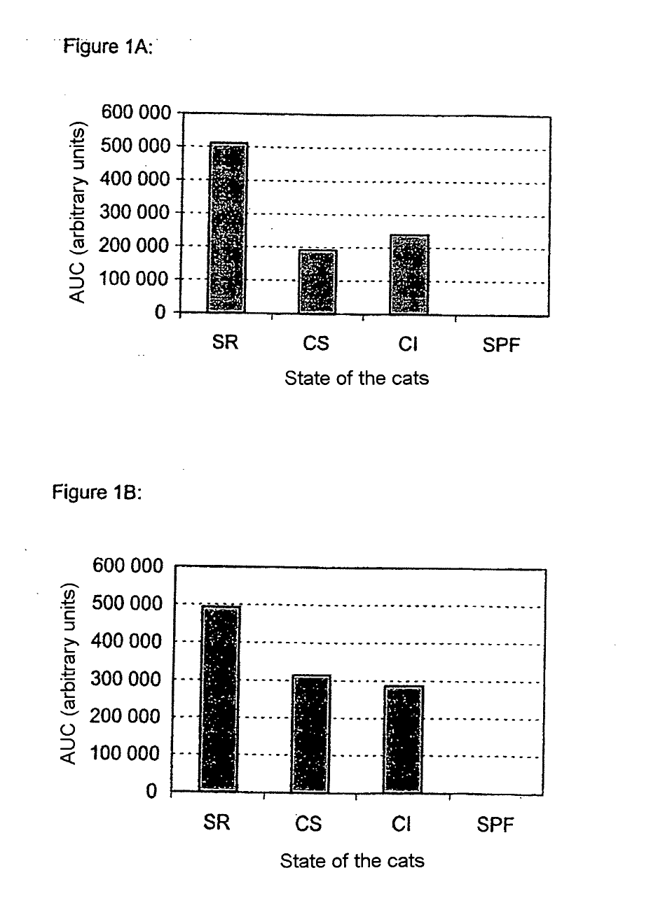

FIG. 1A and 1B: these figures illustrate the reactivity of the peptides T12 and 7I, with the following different categories of sera: SR=spontaneously regressing cats, CS=cats with clinical signs, CI=chronically infected cats, SPF=specific pathogen free cats (control). Densitometric evaluations of the area under the curve (AUC) in the Western blots are given in arbitrary units. The data represent the mean values (n) of the number of serum samples obtained from the respective groups of cats; FIG. 1A: antibody response with respect to T12; FIG. 1B: antibody response with respect to 7I.

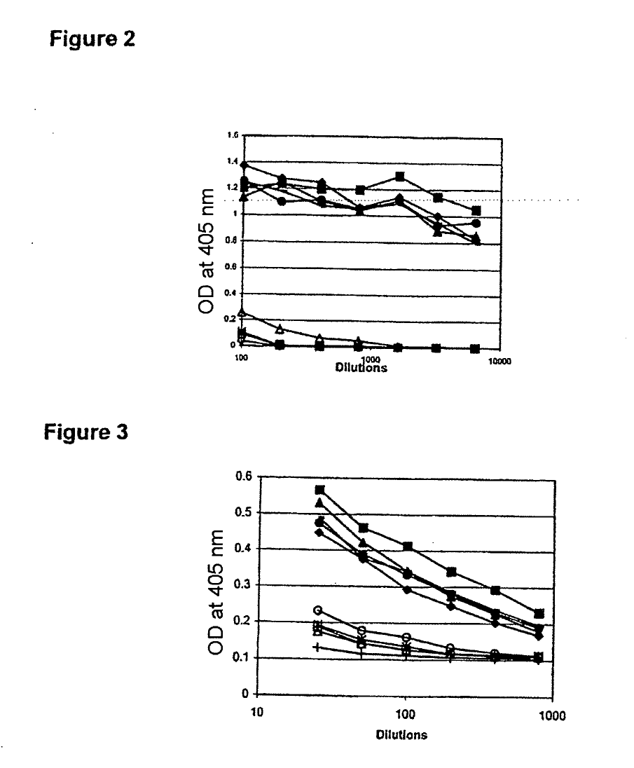

FIG. 2 illustrates the antibody responses against T12 before challenge with the FIPV 79-1146 strain. The five cats vaccinated with the T12 vaccine are represented by the following “solid” symbols ▪, ▴, ●, +, ♦. The five non vaccinated cats are represented by the following “open” symbols □, Δ, ◯, +, ⋄.

FIG. 3 illustrates the antibody responses against the viral antigen (FIPV 79-1146) before challenge with FIPV 79-1146. The five cats vaccinated with the T12 vaccine are represented by the following “solid” symbols ▪, ▴, ●, +, ♦. The five non vaccinated cats are represented by the following “open” symbols □, Δ, ◯, +, ⋄.

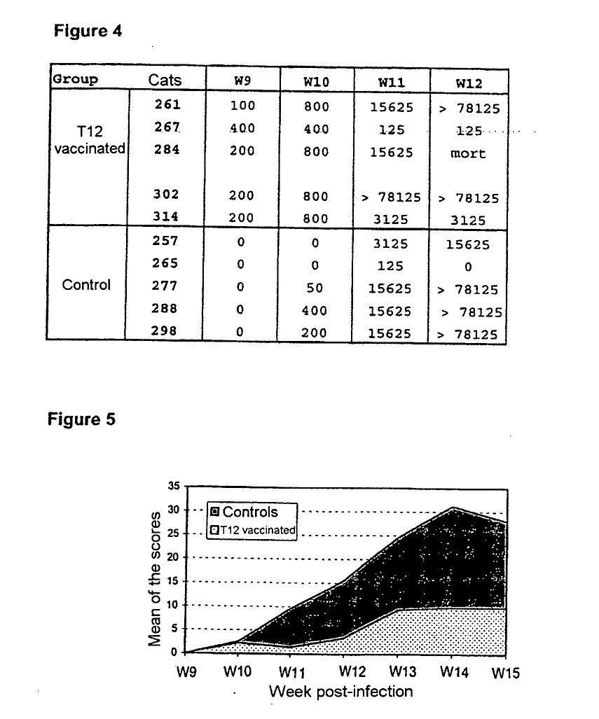

FIG. 4 represents the antibody responses directed against the viral antigen (FIPV 79-1146), after challenge with the FIPV 79-1146 strain.

FIG. 5 represents an evaluation over time of the clinical signs following challenge with FIPV 79-1146. The SPF cats (5 per group) were vaccinated subcutaneously twice at an interval of three weeks with the T12 subunit vaccine, whereas the control cats (5 per group) received the PBS placebo. The two groups of cats were infected with FIPV 79-1146 for six weeks after the second vaccination and the clinical signs were evaluated weekly.

It should be understood, however, that these examples are given solely by way of illustration of the subject of the invention and do not constitute in any manner a limitation thereto.

### Example 1

### Method for Selecting the Peptides According to the Invention

Characteristics of the Immunological Responses in Cats Infected with a Coronavirus

The experiment initially involved 150 cats infected with the feline coronavirus with a known or unknown history of the clinical signs, which were then reduced to 42 cats for the experimental period which lasted for more than one year, leading to the collection of 133 serum samples belonging to the categories CS (42), CI (48), and SR (43). Western blot analysis of the viral proteins M, N and S showed that the three categories of sera revealed a distinct pattern of antigenic reactivity (Table I).

These data show that regression is correlated with the anti-S antibody response; in addition, a high anti-S antibody/anti-M antibody ratio is also observed. On the other hand, the progression of the disease or of the chronic infection is characterized by a lower anti-S/anti-M antibody ratio. The data also indicate that the antibody responses against M and also N proteins are generally dominant compared with the responses against the S protein and have low indicative values as regards the status of the disease. The data also show that it should be technically possible to detect the protective epitope(s) residing in the S protein by Western blotting with SR antisera. These epitopes are scarcely identified or are not identified by CS or CI sera.

TABLE I
Distribution of the immune responses directed
against the various protein components of the virus

| Antigen/ | | | | [S/M] |
|---|---|---|---|---|
| Categories | M | N | S | (×100) |
| SR | 35% | 37% | 20% | 57.1 |
| CS | 67% | 30% | 2% | 3.0 |
| CI | 59% | 35% | 2% | 3.4 |

Identification of the Epitope(s) Involved in the Protective Immune Responses

Using the antisera obtained from the SR cats, the appropriate epitopes were selected with the aid of a random peptide library constructed from the viral genome of the FIPV 79-1146 strain (transcribed and cleaved with a DNase); the fragments obtained are introduced into a bacterial expression system (NovaTope). The fragments of the S gene which were randomly obtained (50-150 base pairs) were isolated by agarose gel electrophoresis and inserted into the plasmid vector (pSCREEN-1b) by the dA-dT ligation method.

Competent E. coli cells (DE3) were then transformed with the recombination vector, the result of which are transformants of the order of 10⁸ cfu/μg of DNA.

The immunoselection of the colonies, performed on NC (nitrocellulose) membranes with 4 different SR sera at a dilution of 1/1000 led to the selection of at least 20 candidate clones which were then subjected to sequencing of their DNA.

The alignments of the inserts with the DNA sequence of the FIPV 79-1146 S gene [De Groot, R. J. et al., 1987] allowed the identification, inter alia, of three candidate peptides respectively called 7I (SEQ ID NO: 2), T12 (SEQ ID NO: 3) and 14I (SEQ ID NO: 4) (Table II).

The two peptides 7I and T12 share an identical region of seven amino acids. The C-terminal region of the S protein comprising the three peptides is highly conserved between the feline coronaviruses serotypes I and II and also among the canine and porcine coronaviruses [Wesseling, J. G. et al., 1994]. It is therefore highly probable that the peptides, if they are immunogenic in cats, are also immunogenic in canine, porcine and human pathologies.

To confirm the positive immunoselection obtained and in order to characterize the protective epitopes, the two peptides called 7I and T12 were expressed by E. coli, in the form of fusion proteins forming inclusion bodies, and extracted from the cell lysate using 8 M urea; after polyacrylamide gel electrophoresis, said peptides were transferred onto a nylon membrane and incubated with the various categories of immune sera, as defined above: SR, CS and CI.

As represented in FIG. 1 (A and B), the peptides 7I and T12 are preferentially identified by SR sera, whereas they are substantially less identified by the CS or CI sera.

TABLE II

Identity of the sequences of the peptides
preferentially identified by the sera of SR
(for spontaneously regressing) cats

| | | |
|---|---|---|
| | | Align- |
| SEQ. | Size | ment (S |

| | | | | |
|---|---|---|---|---|
| ID. | Name | (aa) | Sequence | protein*) |

| | | | | |
|---|---|---|---|---|
| No. 2 | 7I | 21 | GGSWLGGLKDILPSH NSKRKY | 940-960 |
| No. 3 | T12 | 58 | HNSKRKYGSAIEDLLFDKVVTSGLG | 954-1012 |
| | | | TVDEDYKRCTGGYDIADLVCAQYYN | |
| | | | GIMVLPGVA | |
| No. 4 | 14I | 31 | MYQPRVATSSDFVQIEGCDVLFVNATVIDLP | 1274-1304 |

*Numbered according to the order of the sequence of 1452 amino acids of the S protein (SEQ ID NO: 6) (MW = 160, 491) of FIPV 79-1146 [De Groot, R. J. et al., 1987].

### EXAMPLE 2

### Preparation of the Vaccine Composition

Once purified or partially purified, T12 was used as subunit vaccine in a virulent challenge performed on cats. This vaccine exhibits a protective efficacy, which is significant, from the second week after the virulent challenge, in terms of morbidity, severity of the FIP symptoms and particularly in terms of mortality.

The T12 peptide, expressed in the E. coli IPTG induction system, in the form of a recombinant fusion protein (MW≈34 kDa) containing an His6 tag at the C-terminus was extracted from the cell lysate in 8 M urea and purified on a column of Nickel resin (Qiagen). The purified T12 was then adsorbed on aluminum hydroxide (alhydrogel) and formulated in a phosphate buffer (PBS, pH 7.2) and in the presence of saponin (QS21), as adjuvant.

A vaccine dose contains, for a volume of 1.0 ml, 100 μg of T12, 10% of alhydrogel (v/v), and 20 μg of QS21, per cat.

### EXAMPLE 3

### Study of a Vaccination with T12, Followed by a Virulent Challenge

In a group of five SPF cats (HARLAN USA), 8 to 9 weeks old, each animal received two injections subcutaneously, at an interval of three weeks (W0 and W3). In parallel, five control cats (SPF) receive a placebo containing no protein.

Six weeks (W9) after the second vaccination, the two groups of cats were infected oronasally (half orally and the other half nasally) with 220 TCID₅₀ of the FIPV 79-1146 strain. The animals were monitored weekly, for 8 weeks after the challenge, for the determination of the antibody titers (anti-T12 and anti-FIPV) and the clinical signs (morbidity, appearance of the mucous membranes, peritoneal fluid, weight, temperature, hematocrit, leukocytes and mortality).

The clinical signs were measured according to the scheme described in Table III.

TABLE III
Scheme for awarding the points according to
the appearance and the intensity of the
clinical signs

| Categories | Signs | Score | Categories | Signs | Score |
|---|---|---|---|---|---|
| Behavior | Normal | 0 | Loss of | >20% | 1 |
| | Tired | 1 | weight | >30% | 2 |
| | Very tired | 2 | | >50% | 3 |
| | Prostrate | 3 | | | |
| Appearance | Normal | 0 | Body | 39.4-39.9 | 1 |
| of the | Light | 1 | temperature | 40-40.5 | 2 |
| mucous | yellow | | (° C.) | | |
| membranes | Yellow | 2 | | >40.6 | 3 |
| | (lemon) | | | | |
| Peritoneal | Absent | 0 | Hematocrit* | >25% | 0 |
| fluid | Suspicion | 1 | | 25-20% | 1 |
| | Present | 2 | | <20% | 2 |
| Loss of | | 1 | Number of | Reduction | 3 |
| balance** | | | leukocytes | of 50% | |
| Hepatic | | 1 | | | |
| lesions*** | | | | | |
| (postmortem) | | | | | |

*Normal cat hematocrit: of the order of 25 to 45%
**Loss of balance: psychomotor state of the animal; a loss of balance indicates cerebral impairment.
***Hepatic lesions: presence of granulomas on the lobes.

1. Humoral Responses Observed Before the Challenge:
Anti-T12 response: all the vaccinated animals developed a high anti-T12 antibody response, reaching the maximum (Elisa titer>100 000) at approximately 3 weeks (W6) after the second vaccination and remaining relatively stable throughout the period of observation (up to the day of the challenge). On the other hand, it is not possible to detect anti-T12 antibodies (FIG. 2) in the control cats. By Western-blot analysis, the sera of the vaccinated cats showed that they recognized the viral S protein.
Anti-virus response: the cats vaccinated with the T12 peptide developed anti-FIPV responses with antibody titers (by ELISA) ranging from 100 to 200 at W6, increasing slightly at W9, whereas none of the cats of the control group showed detectable responses (FIG. 3).
2. Humoral Responses Developed After the Virulent Challenge
Anti-virus response: Four of the five vaccinated cats responded to the challenge by a rapid rise in the anti-FIPV antibody titer, reaching 5000 to 11,000 at 2 weeks (W11) post-infection. Two of the four responding cats continued to develop higher titers at W12 and at W13 (of about 80,000); whereas one cat remained stable with the titer of 5000 and the other died at W12. The animal which did not respond showed decreased antibody titers.

Four of the five cats of the control group also responded to the challenge with high antibody responses one to two weeks later than the vaccinated cats, the titers reaching levels of 80,000 at W12 and/or at W13. The non responding cat proved transiently seropositive at point W11 with an antibody titer of about 100 (FIG. 4).

3. Monitoring of the Clinical Signs During the Challenge:

The animals were monitored daily: the clinical signs and points were awarded for each observation, on the scale from 0 for normal values to 3 for abnormal values and according to the severity (see Table III). As shown in Table IV, four of the five vaccinated cats (or 80%) did not present or weakly presented clinical signs, whereas one of the cats developed the clinical signs typical of FIP.

Unlike the vaccinated group, three of the five animals of the control group exhibited the clinical signs characteristic of FIP, leading as far as death; one animal showed relatively milder symptoms, whereas the last (animal transiently seropositive at the second week) showed no clinical signs.

These data show that the T12 subunit vaccine is capable of conferring protection on cats infected with a virulent coronavirus (no appearance of enhancing antibodies).

By evaluation of the mortality, the vaccine showed an 80% survival efficacy, compared with 40% in the control group. Among the survivors, some vaccinated animals were completely or almost totally free of symptoms, whereas one of the two surviving control cats developed mild symptoms.

Furthermore, monitoring of the clinical signs over time suggests that the protection induced by the vaccine starts to appear from the second week (W11) post-infection (FIG. 5).

TABLE IV
Total scores observed over the first six
weeks of infection

| | | | | | | |
|---|---|---|---|---|---|---|
| | | | Number | | | Total |
| Cat | Tem- | Weight | of | | Symptom | Score |
| Number | perature | loss | leukocytes | Hematocrit | (**) | (*) |

T12 vaccinated

| | | | | | | |
|---|---|---|---|---|---|---|
| 261 | 0 | 0 | 6 | 1 | 0 | 7 |
| 267 | 0 | 0 | 0 | 0 | 0 | 0 |
| 284 | 0 | 0 | 6 | 2 | 158 | 166* |
| 302 | 0 | 0 | 6 | 0 | 0 | 6 |
| 314 | 0 | 0 | 0 | 0 | 0 | 0 |

Non vaccinated

| | | | | | | |
|---|---|---|---|---|---|---|
| 257 | 0 | 0 | 12 | 7 | 186 | 205* |
| 265 | 0 | 0 | 0 | 0 | 0 | 0 |
| 277 | 1 | 1 | 6 | 3 | 159 | 170* |
| 288 | 2 | 2 | 6 | 2 | 132 | 144* |
| 298 | 1 | 0 | 9 | 0 | 29 | 39 |

*Cats dead with symptoms characteristic of FIP at the 5th or 6th week post-infection.
**Addition of the scores observed for behavior, appearance of the mucous membranes, presence of peritoneal fluid, loss of balance and hepatic lesions.

In general, the values of the scores are between 0 and 3; for certain criteria, the presence (1) or the absence (0) of the criterion is simply noted. In Table IV, the total scores are an addition of the scores which were noted each week, after the challenge. A total of 7 corresponds to the sum: W9=0, W10=1, W11=2, W12=2, W13=2, W14=0 (because the cat died).

Bibliography

1. Addie, D. D. et al., (1992a) Vet.Rec. 130:133. A study of naturally occurring feline coronavirus infections in kittens.
2. Addie, D. D. et al., (1992b) Vet.Rec. 131:202. Feline coronavirus antibodies in cats.
3. Barlough, J. E. et al., (1985) Can.J.Comp.Med. 49:303-307. Experimental inoculation of cats with human coronavirus 229E and subsequent challenge with feline infectious peritonitis virus.
4. Corapi, W. V. et al., (1995) J.Virol. 69(5): 2858-2862. Localization of antigenic sites of the S glycoprotein of Feline Infectious Peritonitis Virus involved in neutralization and antibody-dependent enhancement.
5. De Groot, R. J. et al., (1987) J.Gen.Virol. 68:2639-2646. cDNA cloning and sequence analysis of the gene encoding the peplomer protein of feline infectious peritonitis virus.
6. Gonon, V. et al. (1999) J.Gen.Virol. 80:2315-2317. Clearance of infection in cats naturally infected with feline coronaviruses is associated with an anti-S glycoprotein antibody response.
7. Herrewegh, A. A. P. M. et al., (1998) J.Virol. 72:4508-4514. Feline coronavirus type II strains 79-1683 and 79-1146 originate from a double recombination between feline coronavirus type I and canine coronavirus.
8. Hohdatsu, T. et al., (1992) J.Vet.Med.Sci. 54:557. The prevalence of types I and II feline coronavirus infections in cats.
9. Motokawa, K. et al., (1996). Microbiol. Immunol. 40:425-433. Comparison of the amino acid sequence and phylogenetic analysis of the peplomer, integral membrane and nucleocapsid proteins of feline, canine and porcine coronaviruses.
10. Olsen, C. W. et al., (1992) J.Virol. 66:956-965. Monoclonal antibodies to the spike protein of feline infectious peritonitis virus mediate antibody-dependent enhancement of infection of feline macrophages.
11. Pedersen, N. C. (1976) Feline Pract. 6: 42. Feline infectious peritonitis: Something old, something new.
12. Pedersen, N. C. (1995) Feline Pract. 23:7-20. An overview of feline enteric coronavirus and infectious peritonitis virus infections.
13. Pedersen, N. C. (1988) In: N. C. Pedersen (Editor), Feline infectious diseases, American Veterinary Publications, Santa Barbara, Calif., pp.45-59. Feline infectious peritonitis.
14. Pedersen, N. C. et al. (1983) Am.J.Vet.Res. 44:229-234. Attempted immunization of cats against feline infectious peritonitis, using avirulent live virus or sublethal amounts of virulent virus.
15. Pedersen, N. C. et al. (1980) Am.J.Vet.Res. 41:868-876. Immunologic phenomena in the effusive form of feline infectious peritonitis.
16. Pedersen, N. C. et al. (1985) Comp.Cont.Edu. 7:1001-1011. Experimental studies with three new strains of feline infectious peritonitis virus: FIPV-UCD2, FIPV-UCD3, and FIPV-UCD4.
17. Pedersen, N. C. et al., (1983) In: Molecular Biology and Pathogenesis of Coronaviruses, Plenum Press, New York, pp.365-380. Pathogenic differences between various feline coronavirus isolates.
18. Poland, A. M. et al., (1996) J.Clin.Microbiol. 34:3180-3184. Two related strains of feline infectious peritonitis virus isolated from immunocompromised cats infected with a feline enteric coronavirus.
19. Stoddart, C. A. et al. (1988) Res.Vet.Sci. 45:383-388. Attempted immunization of cats against feline infectious peritonitis using canine coronavirus.
20. Vennema, H. et al., (1991) Virology 181: 327-335. Primary structure of the membrane and nucleocapsid protein genes of feline infectious peritonitis virus and immunogenicity of recombinant vaccinia viruses in kittens.
21. Vennema, H. et al. (1995) Feline Pract. 23: 40-44. A comparison of the genomes of FeCVs and FIPVs and what they tell us about the relationships between feline coronaviruses and their evolution.
22. Wesseling, J. G. et al., (1994) J.Gen.Virol.75: 1789-1794. Nucleotide sequence and expression of the spike (S) gene of canine coronavirus and comparison with the S proteins of feline and porcine coronaviruses.
23. Woods, R. D. and Pedersen, N. C. (1979) Vet.Microbiol. 4:11-16. Cross protection studies between feline infectious peritonitis and porcine transmissible gastroenteritis.

1-23. (Cancelled).

24. A peptide, which is selected from the group consisting of SEQ ID NO: 3, from 12 to 20 amino acids of SEQ ID NO: 3, SEQ ID NO: 4, from 12 to 20 amino acids of SEQ ID NO: 4, the peptide SEQ ID NO: 5 and from 12 to 20 amino acids of SEQ ID NO: 5.

25. A modified peptide, which corresponds to the peptide as claimed in claim 24, into which there have been introduced artificial mutations, deletions, insertions, variations or combinations of these events, provided that the peptides thus modified do not induce enhancement phenomena.

26. The peptide as claimed in claim 24, whichs is in the form of a synthetic peptide, a repeated peptide or a protein fused at the N- or C-terminal end with a protein of another feline pathogenic agent.

27. A method of preventing or protecting a cat against feline infections peritonitis (FIP), wherein the method comprises administering to the cat an amount of a peptide according to claim 24 sufficient for inducing an exclusively neutralizing protection against feline infectious peritonitis.

28. A vaccine for protecting cats against feline infectious peritonitis (FIP), which comprises at least one peptide as claimed in claim 24, in combination with at least one of a carrier substance, an adjuvant, and at least one pharmaceutically acceptable vehicle.

29. The vaccine as claimed in claim 28, comprising an adjuvant; wherein the adjuvant is selected from the group consisting of an oily emulsion, saponin, an inorganic substance, a bacterial extract, aluminum hydroxide, squalene, and mixtures thereof.

30. The vaccine as claimed in claim 28, comprising a carrier substance; wherein the carrier substance is selected from the group consisting of a unilamellar liposome, a multi lamellar liposome, a saponin micelle, a solid micro sphere of a saccharide, a solid microsphere of auriferous nature, and mixtures thereof.

31. The vaccine as claimed in claim 28, which further comprises other viral proteins or peptides.

32. A nucleic acid molecule encoding a peptide as claimed in claim 24.

33. The nucleic acid molecule as claimed in claim 32, which is selected from the group consisting of SEQ ID NO: 7, SEQ ID NO: 8, SEQ ID NO: 9, from 36 to 60 nucleotides of SEQ ID NO: 8 and from 36 to 60 nucleotides of SEQ ID NO: 9.

34. The use of at least one nucleic acid molecule as claimed in claim 32 for the construction of recombinant vectors, which are useful as vaccines.

35. A method of preventing or protecting a cat against feline infectious peritonitis (FIP), wherein the method comprises administering to the cat an amount of a nucleic acid according to claim 32 sufficient for inducing an exclusively neutralizing protection against feline infectious peritonitis.

36. A recombinant vector, wherein said vector comprises a nucleic acid molecule as claimed in claim 32.

37. The vector as claimed in claim 36, wherein said vector is a viral vector selected from the group consisting of poxviruses, adenoviruses, retroviruses, and herpes viruses; a bacterial vector selected from the group consisting of mycobacteria, enterobacteria, and lactobacilli; or a plasmid comprising a sequence encoding at least one of peptide which is selected from the group consisting of SEQ ID NO: 3, from 12 to 20 amino acids of SEQ ID NO: 3, SEQ ID NO: 4, from 12 to 20 amino acids of SEQ ID NO: 4, the peptide SEQ ID NO: 5 and from 12 to 20 amino acids of SEQ ID NO: 5.

38. A vaccine for protecting cats against feline infectious peritonitis (FIP), wherein said vaccine comprises a recombinant vector as claimed in claim 36 or at least one nucleic acid molecule encoding a peptide that is selected from the group consisting of the SEQ ID NO: 3, from 12 to 20 amino acids of SEQ ID NO: 3, SEQ ID NO: 4, from 12 to 20 amino acids of SEQ ID NO: 4, SEQ ID NO: 5 and from 12 to 20 amino acids of SEQ ID NO: 5.

39. A vaccine for protecting cats against feline infectious peritonitis (FIP), wherein said vaccine comprises a recombinant vector as claimed in claim 37 and at least one nucleic acid molecule wherein at least one nucleic acid molecule is selected from the group consisting of SEQ ID NO: 7, SEQ ID NO: 8, SEQ ID NO: 9, from 36 to 60 nucleotides of SEQ ID NO: 8 and from 36 to 60 nucleotides of SEQ ID NO: 9.

40. A method for selecting immunogenic peptides corresponding to a fragment of a coronavirus S protein, and not inducing enhancement phenomena, which comprises:
constructing a random peptide library corresponding to a coronavirus S protein fragment, from at least one FIPV viral genome,
bringing said peptides into contact with at least four different spontaneously regressing (SR) cat sera, after infection with FIPV, at a dilution of at least 1/1000, and
immunoselecting the peptides interacting with said spontaneously regressing cat sera, but scarcely or not even interacting at all with sera from cats exhibiting clinical symptoms (CS) or sera from cats exhibiting subclinical signs of chronic infection (CI) with FIPV.
